(19) United States
(12) Patent Application Publication
(10) Pub. No.: US 2024/0360066 A1
(43) Pub. Date: Oct. 31, 2024

(54) Process for alkoxycarbonylation of olefins using synthesis gas

(71) Applicant: Evonik Oxeno GmbH & Co. KG, Marl (DE)

(72) Inventors: Robert Franke, Marl (DE); Matthias Beller, Ostseebad Nienhagen (DE); Peter Kucmierczyk, Herne (DE); Ralf Jackstell, Rostock (DE); Yao Ge, Chicago, IL (US); Helfried Neumann, Rostock (DE)

(73) Assignee: Evonik Oxeno GmbH & Co. KG, Marl (DE)

(21) Appl. No.: 18/638,013

(22) Filed: Apr. 17, 2024

(30) Foreign Application Priority Data
Apr. 26, 2023 (EP) 23170052.7

(51) Int. Cl. C07C 67/38; B01J 31/22; B01J 31/24

(52) U.S. Cl. CPC C07C 67/38; B01J 31/2234; B01J 31/2409; B01J 2231/321; B01J 2531/004; B01J 2531/824

(57) ABSTRACT

Process for alkoxycarbonylation of olefins using synthesis gas.

The invention relates to a process for alkoxycarbonylation of olefins using synthesis gas (=mixture of CO and H₂).

The alkoxycarbonylation of ethylenically unsaturated compounds is a process of increasing significance. An alkoxycarbonylation is understood to mean the reaction of ethylenically unsaturated compounds (olefins) with carbon monoxide and alcohols in the presence of a metal-ligand complex to give the corresponding esters. Typically, the metal used is palladium. The following scheme shows the general reaction equation for an alkoxycarbonylation:

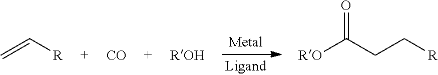

Conventionally, an alkoxycarbonylation process is operated using CO. One is described in EP 4 001 256 A1. The feeding of H₂ has to date been deliberately avoided since it was assumed that this would promote side reactions, for example a hydroformylation, and hence the conversion to the desired target product would decline.

The technical object of the invention is that of providing a novel process that delivers an increased yield.

The object is achieved by a process according to Claim 1.

Process comprising the process steps of:
a) initially charging an olefin;
b) adding a ligand of formula (I):

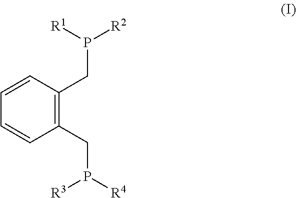

where
R¹ and R³ are each a —(C₃-C₂₀)-heteroaryl radical,
R² and R⁴ are each —(C₁-C₁₂)-alkyl;
c) adding a compound comprising Pd;
d) adding an alcohol;
e) feeding in CO and H₂, where the pressure with which H₂ is fed in is at least 0.6 MPa (6 bar);
f) heating the reaction mixture from steps a) to e), to convert the olefin to an ester.

The expression (C₁-C₁₂)-alkyl encompasses straight-chain and branched alkyl groups having 1 to 12 carbon atoms. These are preferably (C₁-C₈)-alkyl groups, more preferably (C₁-C₆)-alkyl, most preferably (C₁-C₄)-alkyl.

The expression (C₃-C₂₀)-heteroaryl encompasses mono- or polycyclic aromatic hydrocarbon radicals having 3 to 20 carbon atoms, where one or more of the carbon atoms are replaced by heteroatoms. Preferred heteroatoms are N, O and S. The (C₃-C₂₀)-heteroaryl groups have 3 to 20, preferably 6 to 14, particularly preferably 6 to 10 ring atoms. Thus, for example, pyridyl is in the context of this invention a C₆-heteroaryl radical and furyl is a C₅-heteroaryl radical.

In one variant of the process, CO is fed in with a pressure in the range from 1 MPa (10 bar) to 3 MPa (30 bar).

In one variant of the process, CO is fed in with a pressure in the range from 1.5 MPa (15 bar) to 2.5 MPa (25 bar).

In one variant of the process, CO is fed in with a pressure of 2 MPa (20 bar).

In one variant of the process, H₂ is fed in with a pressure in the range from 0.6 MPa (6 bar) to 2.9 MPa (29 bar).

In one variant of the process, H₂ is fed in with a pressure in the range from 0.9 MPa (9 bar) to 2.1 MPa (21 bar).

In one variant of the process, H₂ is fed in with a pressure in the range from 1 MPa (10 bar) to 2 MPa (20 bar).

In one variant of the process, the ratio of the pressures with which CO and H₂ are fed in is in the range from 1:0.3 to 1:1.4.

In one variant of the process, R¹, R³ are each selected from furyl, thienyl, pyrrolyl, oxazolyl, isoxazolyl, thiazolyl, isothiazolyl, imidazolyl, pyrazolyl, furazanyl, tetrazolyl, pyridyl, pyridazinyl, pyrimidyl, pyrazinyl, benzofuranyl, indolyl, isoindolyl, benzimidazolyl, quinolyl, isoquinolyl.

In one variant of the process, R² and R⁴ are ^{ter}Bu.

In one variant of the process, the ligand in process step b) has the formula (1):

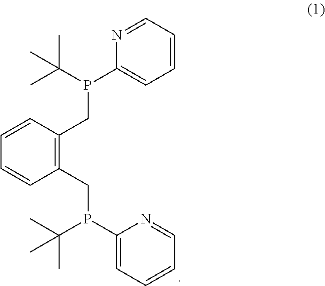 [SMILES: CC(C)(C)P(Cc1ccccc1CP(c1ccccn1)C(C)(C)C)c1ccccn1]

In one variant of the process, the compound in process step c) comprising Pd is selected from palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), (cinnamyl)palladium dichloride.

In one variant of the process, the compound in process step c) comprising Pd is Pd(acac)₂.

In one variant of the process, the alcohol in process step d) is selected from methanol, ethanol, 1-propanol, 1-butanol, 1-pentanol, 1-hexanol, 2-propanol, tert-butanol, 3-pentanol, cyclohexanol, phenol, or mixtures thereof.

In one variant of the process, the alcohol in process step d) is methanol.

In one variant of the process, the process comprises the additional process step d′) of: d′) adding an acid selected from: aluminium triflate, sulfuric acid, methylsulfonic acid (MSA), para-toluenesulfonic acid (p-TSA).

In one variant of the process, aluminium triflate is added in process step d′).

In one variant of the process, the acid:ligand ratio is in the range from 1 mol:1 mol to 15 mol:1 mol.

In one variant of the process, the aluminium triflate:ligand ratio is in the range from 1 mol:1 mol to 15 mol:1 mol.

The reaction mixture is heated in process step f) of the process according to the invention preferably to a temperature in the range from 30° C. to 150° C., preferably from 40° C. to 140° C., more preferably from 50° C. to 120° C., in order to convert the olefin to an ester.

The invention is to be elucidated in detail hereinafter with reference to a working example.

### Experiment 1

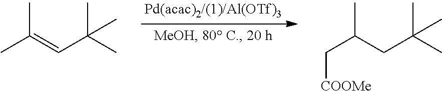 [0039] Pd (acac)₂: 0.04 mol %
(1): 0.12 mol %
Al(OTf)₃: 0.6 mol %

The experiment was conducted once with synthesis gas and, as a comparative experiment, with pure CO. The synthesis gas here was a mixture of CO and H₂. In both experiments, CO was fed in with a pressure of 20 bar. In the experiment with synthesis gas, the H₂ was likewise fed in at 20 bar, which established a total pressure of 40 bar.

Yield of ester:
CO: 47%
CO+H₂:67%

### Experiment 2 (Variation of H₂ Pressure)

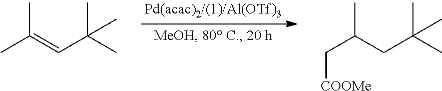 [0047] Pd (acac)₂: 0.04 mol %
(1): 0.12 mol %
Al(OTf)₃: 0.6 mol %

The series of experiments was conducted with a constant CO pressure of 20 bar. The H₂ pressure was varied according to the table below.

| p(H₂) [bar] | Yield [%] |
|---|---|
| 0 | 47 |
| 5 | 44 |
| 10 | 63 |
| 20 | 67 |
| 30 | 41 |
| 40 | 41 |

As shown by the experiments conducted, the yield was increased by the use of synthesis gas.

1. Process comprising the process steps of:
a) initially charging an olefin;
b) adding a ligand of formula (I): 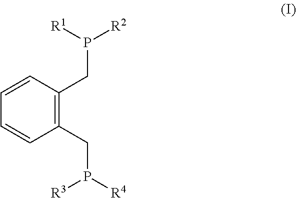
where
R¹ and R³ are each a —(C₃-C₂₀)-heteroaryl radical,
R² and R⁴ are each —(C₁-C₁₂)-alkyl;
c) adding a compound comprising Pd;
d) adding an alcohol;
e) feeding in CO and H₂, where the pressure with which H₂ is fed in is at least 0.6 MPa (6 bar);
f) heating the reaction mixture from steps a) to e), to convert the olefin to an ester.

2. Process according to claim 1,
wherein CO is fed in with a pressure in the range from 1 MPa (10 bar) to 3 MPa (30 bar).

3. Process according to claim 1,
wherein CO is fed in with a pressure in the range from 1.5 MPa (15 bar) to 2.5 MPa (25 bar).

4. Process according to claim 1,
wherein H₂ is fed in with a pressure in the range from 0.6 MPa (6 bar) to 2.9 MPa (29 bar).

5. Process according to claim 1,
wherein H₂ is fed in with a pressure in the range from 0.9 MPa (9 bar) to 2.1 MPa (21 bar).

6. Process according to claim 1,
wherein the ratio of the pressures with which CO and H₂ are fed in is in the range from 1:0.3 to 1:1.4.

7. Process according to claim 1,
where R¹, R³ are each selected from furyl, thienyl, pyrrolyl, oxazolyl, isoxazolyl, thiazolyl, isothiazolyl, imidazolyl, pyrazolyl, furazanyl, tetrazolyl, pyridyl, pyridazinyl, pyrimidyl, pyrazinyl, benzofuranyl, indolyl, isoindolyl, benzimidazolyl, quinolyl, isoquinolyl.

8. Process according to claim 1,
where R² and R⁴ are ^{ter}Bu.

9. Process according to claim 1,
wherein the ligand in process step b) has the formula (1):

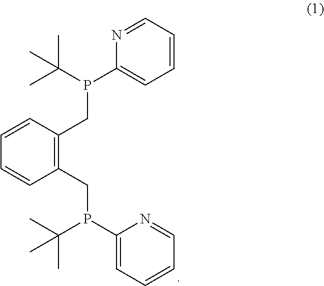 [SMILES: CC(C)(C)P(Cc1ccccc1CP(c1ccccn1)C(C)(C)C)c1ccccn1]

10. Process according to claim 1,
wherein the compound in process step c) comprising Pd is selected from palladium dichloride, palladium(II) acetylacetonate, palladium(II) acetate, dichloro(1,5-cyclooctadiene)palladium(II), bis(dibenzylideneacetone)palladium, bis(acetonitrile)dichloropalladium(II), (cinnamyl)palladium dichloride.

11. Process according to claim 1,
wherein the compound in process step c) comprising Pd is Pd(acac)₂.

12. Process according to claim 1,
wherein the alcohol in process step d) is selected from methanol, ethanol, 1-propanol, 1-butanol, 1-pentanol, 1-hexanol, 2-propanol, tert-butanol, 3-pentanol, cyclohexanol, phenol, or mixtures thereof.

13. Process according to claim 1,
wherein the alcohol in process step d) is methanol.

14. Process according to claim 1,
wherein the process comprises the additional process step d′) of:
d′) adding an acid selected from: aluminium triflate, sulfuric acid, methylsulfonic acid (MSA), para-toluenesulfonic acid (p-TSA).

15. Process according to claim 14,
wherein the acid:ligand ratio is in the range from 1 mol:1 mol to 15 mol:1 mol.
